(19) United States
(12) Patent Application Publication
(10) Pub. No.: US 2008/0287745 A1
(43) Pub. Date: Nov. 20, 2008

(54) Alarm Device for Preventing Cot Death

(76) Inventor: Thierry Hartmann, Les Molieres (FR)

Correspondence Address: OLIFF & BERRIDGE, PLC, P.O. BOX 320850, ALEXANDRIA, VA, 22320-4850, US

(21) Appl. No.: 11/631,685

(22) PCT Filed: Jul. 8, 2005
(86) PCT No.: PCT/IB2005/001940

§ 371 Date: Feb. 5, 2007

(30) Foreign Application Priority Data
Jul. 9, 2004 (FR) 0407676

(51) Int. Cl. A61B 5/00

(52) U.S. Cl. 600/300

(57) ABSTRACT

The invention relates to a device comprising means for the remote transmission of an active control signal for triggering alarm means (2, 10), for a person looking after a child. Said alarm control signal is generated by the control of sensor means which are sensitive to the spatial orientation of a plate mounted in a support (1) to be fixed to the clothing of the child to be watched. The support (1) is fixed to the navel of the child in such a way that the signal triggering the alarm means is automatically generated when the sensor means detect a variation in the orientation of the plate which is higher than a pre-determined threshold. The support (1) is arranged in a remote unit which paralyses the alarm means.

The present invention relates to a cot death prevention device, intended for warning a person in charge of the supervision of an infant that the latter is turning onto his/her stomach during his/her sleep or during a resting period when he/she is lying down.

Recent studies have shown that infants lying on their stomachs were more stricken by the cot death syndrome than those lying on their backs. It is thus recommended to always put the infant to lie on his/her back and to constantly make sure that the infant does not turn on his/her stomach of his/her own accord. However, this constant supervision is practically impossible to put into practise, especially at night when the persons in charge of the infant's supervision need to sleep in the same capacity as the infant to be supervised.

The invention is purposed, while leaving the infant his/her freedom of movement, to provide the persons in charge of his/her supervision with means of alerting them when the infant turns from the dorsal position to the ventral position, including when they are at a distance from the infant, and, if necessary, waking them up when they are asleep.

The cot death prevention device according to the invention comprises various material means that are advantageously split into two distinct units, separated or at least separable from one another, and that co-operate with one another to automatically detect whether the infant being supervised has moved from the back position enough to risk falling into the front position and to then automatically control the transmission of an alarm signal informing the person in charge of this supervision of the danger. The means in question shall be defined herein and described in their preferred forms of realization, from which the characteristics of the invention best suited to the requirements of the practice will emerge, namely in terms of manufacturing costs and conditions, solidity, convenience of use, cleanliness, safety, performance efficiency, thereby providing a particularly satisfactory solution to the issue of cot death prevention.

In accordance with the preferred characteristics of the device according to the invention, there are means of remote transmission of an alarm control signal that triggers alarm means within the reach of a person in charge of the supervision of an infant, and this alarm control signal is produced under the control of detector means responsive to the orientation in space of a plate mounted in a support to be attached to the clothing of the infant to be supervised. It is advantageously to the area of the infant s navel that the plate support holding the detector means is to be attached, such that the signal triggering the alarm means are automatically generated when the detector means detect that an orientation variation of the plate that is greater than a predetermined threshold has occurred.

The shape and constitution of the plate are not of major importance herein, no more than those of its support, except that the latter should make it easy for the user to place it on the infant in a predetermined arrangement, prescribed for its purpose of preventing cot death. The device must remain on standby, with no effect on the production of an alarm signal when the infant is lying on his/her back, the variations in orientation to which the detector means are responsive first being those of a longitudinal plane through the infant's vertebral column, when it tilts by a value expressed by an angle of angular position variation in a transverse plane thus normally vertical but also perpendicular to the infant's vertebral column.

The detector means, in the simplest manner, consist of an angular position detector that functions in relation to a vertical axis, such as determined, in direction and orientation, by the pull of gravity. The threshold triggering the alarm in the event of an orientation variation corresponding to a tilting of the plate rotatably around its longitudinal horizontal axis is advantageously set to an angular position value at least in the order of 90 degrees in relation to this vertical axis reference, preferentially between 110 and 150 degrees, but possibly up to 180 degrees.

According to the detector means chosen, provision may be advantageously made to further detect any orientation variation of the plate exceeding a predetermined threshold by tilting rotatably around an axis transverse to the infant's vertebral column. Hence namely around the horizontal direction perpendicular to the horizontal axis cited previously. Such arrangements secondly allow to alert the person in charge of the infant in the event that the latter should move into a sitting, or even standing position. The threshold angle of the corresponding angular position is advantageously set to a variation in the order of 90 degrees from back to forth (from the infant's head to feet). If necessary, this second alert will be of a nature different to that of the first, differing for example in intensity or in tone for a sound signal, and the person in charge of the infant could then decide whether the infant is old enough to sit up or if it is necessary to lay the infant back down before he/she topples onto his/her stomach.

The detector means are advantageously provided in a first unit of the device, whereby they are associated with means of attachment to the infant's clothing, which may namely function by clipping to a pyjama belt or his/her nappy. It is on a second unit, usually separate and freely movable independently of the first, that the alarm means are found, preferably incorporated into a alarm case that is movable separately from the first unit, that the person in charge of the infant's supervision may easily carry to place it far from the infant but within his/her own alarm signal reach.

The transmission of the alarm control signal produced by the detector means takes place from a transmitter of said signal that is integral with the first unit to a cooperating receiver that is integral with the second by any known means, but preferably using a wireless remote transmission channel. The signal is thus advantageously carried in the atmosphere in the form of electromagnetic waves of frequencies inaudible to man. The transmitter is controlled by the detector means, the receiver controls the occurrence of the alarm information. This advantageously involves triggering the transmission of an alarm signal aimed at the person in charge of the infant's supervision, namely of the visual and/or sound alert type. It is generally desirable to avoid the effect of external interferences by fitting the device of the invention with a system known per se, which may be of the loop code type to automatically tune the transmitter and receiver on one same frequency, or of the encoder switch type for manual tuning.

Within the framework of a preferred embodiment in accordance with the invention, it is advantageous to provide, in the alarm case of the second unit of the device, a storage space for the first unit that comprises means of hindering the functioning of the detector means, by preventing them from triggering the alarm. With concrete realization of such means, it is namely possible to associate a magnetic control switch arranged on the power supply circuit of the remote control signal transmitter with a magnetic field created by magnet in the space accommodating the first unit in the second. Therein, of course, an electric coil would be equivalent to a magnet.

Other characteristics of the present invention are more in connection with the requirements of a reliable realization that ensures comfort, hygiene and safety for the infant, as well as a functioning that is convenient and permanently safe for the peace of mind of the persons in charge of him/her. This is the case with the characteristic of the invention that consists in enclosing the elements of the first unit (that that is attached to the infant) in a fleece-lined cushion eliminating any risk of injury without hampering the manipulation of the means used to attach it temporarily to the infant's clothing. It is also the case with the realization of such a cushion, externally, to constitute a recreational or clothing decoration object, for example in the shape of a pet.

It is further the case with another special characteristic of the invention, that consists in embedding the constituents and electronic circuits of the first unit in an electrically insulating and sealed mass, as is commonly known using polymer-based plastic materials. In addition to the advantages of solidity, this leads to convenient dismounting of the electronic assembly, whether to replace it, to change the battery, or to wash the cover cushion.

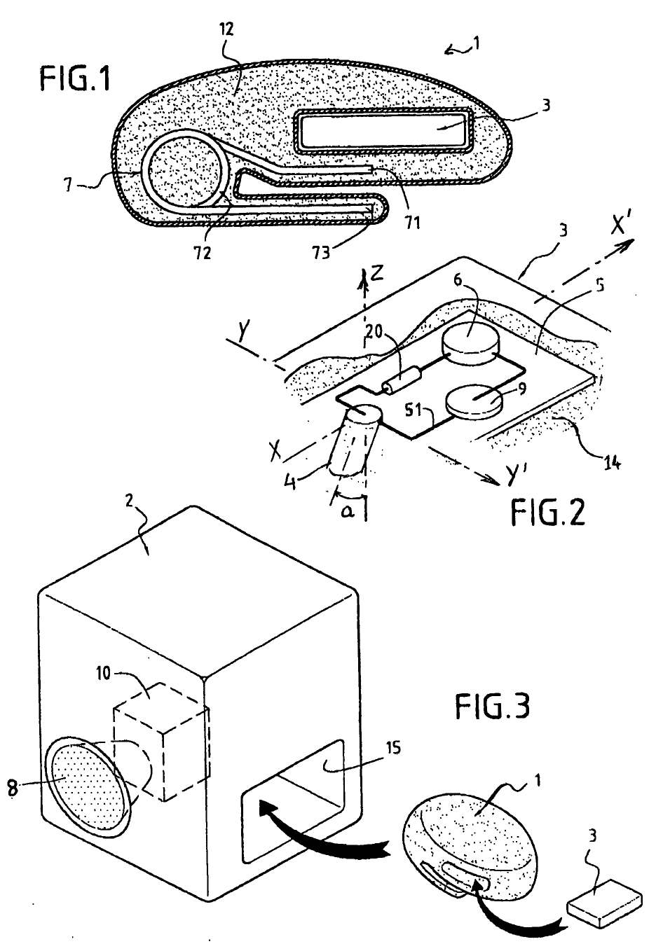

The invention will now be more fully described within the framework of the preferred characteristics and their advantages, with reference to FIGS. 1 to 3, that illustrate a special embodiment of the device according to the invention and among which:

FIG. 1 shows, in a sectional view, a first of the two main units constituting the device, that that is to be attached to the clothing of the infant to be supervised;

FIG. 2 is a schematic diagram illustrating the constitution of the detector means that it comprises;

and FIG. 3 illustrates a particularly advantageous means of storing this first unit in the second unit of the device according to the invention, which is to be placed within the reach of a person in charge of the infant's supervision.

FIG. 3 is a general illustration of a preferred embodiment of the alarm device according to the invention. The device consists of two units, one to be attached to the infant's clothing, the other to be placed proximate to the person in charge of the infant's supervision.

The first unit of the device, with the reference 1 in the Figures, is made to be attached to the clothing of an infant to be supervised. This first unit 1 comprises means 3 made to remotely control the triggering of an alarm 10 included in an alarm case 2. The alarm case 2 constitutes the second unit of the device according to the invention. It is made portable, such that the person in charge of the infant's supervision may conveniently place it nearby.

The unit 1 contains a plate 5, on which an angular position detector 4 is mounted, that is responsive to the orientation of the plate in space and that functions in relation to the vertical direction defined by gravity using as a sensor a weighted element freely movable in a closed room. The plate 5 supports a printed circuit board that associated the detector with a transmitting component 6 to control, according to the position of the sensor element, the transmission by the latter of the alarm control signal. In a manner known per se, the remote transmission of this signal to a cooperating receiver integral with the second unit (alarm case 2) takes place without using a wire connection, thus more specifically by hertzian rays, under frequencies inaudible to man.

The plate further supports a miniature battery 9 ensuring the electrical power supply of the circuit 51 operatively connecting the detector 4 to the transmitter 6, and finally a magnet switch 20, that is interposed on this circuit to disable the assembly and to avoid untimely triggering of the alarm.

In the plate, the different components are embedded in an insulating resin mass 14 that is incorporated, as shown in FIG. 1, into a protective cushion 12. This cushion, designed with ample fleece-lining to eliminate any risk of discomfort or injury for the infant, also envelops a clip 7 serving as means of attaching the unit 1 to the infant's clothing.

This clip has been illustrated in the Figure by a spring 72 elastically drawing two grippers 71 and 73 between which the infant's belt or nappy, for example, may be inserted. In practice, the use of a safety clip of any commercially available model is generally preferable. In all cases, it is provided that in the cushion, the space accommodating the plate 5 is fixed in relation to the fixed gripper of a clip of this type (or any other equivalent means of attachment). This will afford a correct position of the enabled device on the infant, by determining for example, in the case described, that the plate is arranged on a vertical plane when the infant is suitably lying on his/her back. As the detector 4 is fixed on the plate, the vertical reference is thus also determined.

The fixed unit 2 to be placed proximate to the person in charge of the infant is very schematically illustrated in FIG. 3 by a box enclosing the electronic circuits of the alarm device 10, which comprise a receiver of the remote control signals from the first unit that controls the alarm signal transmission. The latter is hereby assumed to be a sound signal, emitted by a loudspeaker 8. If necessary, it may be completed by a visual signal.

It can also be seen in FIG. 3 that the alarm case includes a space 15 for storing the unit 1 when it is not in use. In this space there is a magnetic field that controls the switch 20 of the circuit 51 to remain permanently open. When the cushion of unit 1 is positioned in its space, the system functioning is henceforth hindered, and regardless of the orientation that the plate adopts, the alarm cannot be triggered. The alarm case 2 can easily be made to be easily handled to be used to carry the cushion 12 until it is positioned on the infant when the latter has already been put to bed.

Reference will now be made to FIG. 2 to outline the functioning of the device. In this Figure, a trirectangular reference system of vertical axis Z is shown. On the horizontal plane XY we accept that the axis XX′ is oriented alongside the infant's vertebral column, from his/her feet to his/her head. The axis YY′ is thus horizontal in the transverse direction, perpendicular to the infant's vertebral column, from his/her right to his/her left.

The device is designed to trigger the alarm when the infant turns either in a rightward direction, or in a leftward direction, until he/she is lying on his/her side, with a tendency to move into the ventral position. This is obtained, in accordance with one of the preferred methods of implementation of the invention, using as a detector a level detector model commercially available, essentially comprising a ball made of an electrically conductive material movable in a space forming a spherical cap with conductive coating, that is responsive in all directions to the angular variations exceeding a threshold of 120 degrees.

This detector is fixed in the plate 5. It is embedded therein in a cavity formed in a longitudinal vertical plane which is between the vertical plane of symmetry ZX containing the infant's vertebral column when the unit 1 of the device of the invention is attached to around the area of the infant's navel in a correctly oriented position. Henceforth, it is readily understood that when the infant turns onto one side, in a rightward or in a leftward direction indifferently, he/she is only authorised to do so as long as the detector tilts to the vertical of a remaining angle, on the transverse plane ZY, below the threshold value of 120 degrees, whereas beyond this, the detector ball closes the power supply circuit of the alarm control signal transmitter and the alarm is triggered.

In the longitudinal plane of symmetry of the detector, the cavity that accommodates it is tilted towards the bottom towards the back, that is, from the infant's head to feet. The tilt angle (“a” in FIG. 2) is in the order of 30 degrees, such that in this plane, the alarm is triggered as soon as the vertical tilt angle reaches 90 degrees. As a result, when the infant sits up, the alarm is triggered without waiting for him to lean forward until he/she reaches an angle of 120 degrees.

The invention provides another advantageous form of realization of the angular position detector, of which the advantage in relation to that in FIG. 2 is that it is simpler and cheaper, when less accuracy in the detection thresholds can be used.

According to this alternative case, not illustrated in the drawings, the responsive element of the detector consists of the electric power supply battery of the circuit 51. For that, the battery is movably mounted in a space that is vertical in parallel with the longitudinal axis XX′ of the plate. The battery moves therein under the effect gravity between a bottom of the space in which it remains electrically insulated and an opposite bottom with contacts closing the electrical circuit 51. When the infant moves into the ventral position in a rightward or in a leftward direction, the battery falls to the bottom of its space and closes the electrical circuit, hence triggering the alarm. Generally, for that, the angle of the space must simply have markedly exceeded the horizontal with a variation of 90 degrees, without having to reach the 180 degree position.

As a further alternative, the device according to the invention may be made with a battery supplying the electrical circuits of the first unit made removable, and also easily replaceable (or rechargeable in the case of a battery cell) when its charge level becomes insufficient to ensure the proper functioning of the detection of the risk of cot death.

Moreover, for the brevity of description, the technical details of realization understood by those skilled in the art have not been mentioned. This is the case, for example, with the test means that advantageously complete the electronic circuits of the detector, to inform the user of an electrical power supply failure. When the user is warned in time of the arising of an existing or predictable failure, he/she simply needs to replace the battery if it is removable. But the indication of an existing or potential failure is also very useful if the battery is incorporated with the other electronic components into a matrix of insulating embedding material, in the form of a disposable assembly.

It is clear from the foregoing that the invention is not restricted to the implementation methods specifically described and shown in the drawings.

In particular, the detector is illustrated by realizations according to which the responsive element is a movable mass. As an equivalent solution, a detector of which the functioning is based on the variations of an electrical resistance according to the orientation of the plate can be used. Such a detector may, for example, be associated with a visual alarm signal, produced by light-emitting diodes, of which a number light up according to the degree of the angle of tilt detected.

1-10. (canceled)

11. A cot death prevention device, comprising two units that are movable independently of one another, detector means responsive to the orientation in space of a plate mounted in a support provided in a first one of said units that attaches in a predetermined arrangement to the clothing of the infant to be supervised, means of remote transmission for an alarm control signal that is produced under the control of said detector means and that triggers alarm means incorporated in the second of said units for triggering said alarm means in the event of detection of an orientation variation of said plate that is greater than a predetermined threshold, and further comprising means to hinder the operation of said detector means in producing said alarm control signal when said first unit is in a position of storage in the first unit.

12. A device according to claim 11, wherein said detector means comprise an angular position detector functioning under the effect of gravity in relation to a vertical axis and wherein said support is shaped to facilitate its positioning by attachment to the infant's clothing in an arrangement wherein the angular position detected at least comprises that of the plate in its orientation variations in tilt with respect to a longitudinal plane through the infant's vertebral column.

13. A device according to claim 11, wherein said detector means are associated in said first unit of the device with means of attachment to the clothing of an infant to be supervised in said predetermined arrangement, while said second unit incorporating said alarm means is movable separately from said first unit to put the alarm means within the reach of a person in charge of the infant's supervision.

14. A device according to claim 13, wherein said means of attachment to the infant's clothing function by pinching the belt of a nappy it wears.

15. A device according to claim 11, wherein said second unit s made in the form of a case having a storage space for the first unit in which a magnetic field is adapted to control a magnet switch of an electrical circuit supplying the detector means so as to hinder the functioning of said detector means producing said alarm control signal when said first unit is positioned in said storage space.

16. A device according to claim 14, wherein said first unit is enveloped in a cushion protecting the infant against risks of injury.

17. A device according to claim 11, wherein said alarm control signal is transmitted wirelessly, in the form of electromagnetic waves, from a transmitter integral with said first unit that is controlled by said detector means to a receiver that is integral with said second unit and that triggers the transmission of an alarm signal towards a person in charge of the infant's supervision, said signal being of the visual and/or sound alert type.

18. A device according to claim 11, wherein said detector means are made and arranged for controlling triggering the alarm when the orientation variation of the plate exceeds a first threshold of at least 90 degrees by rotation around a longitudinal axis that is parallel to the infant's vertebral column.

19. A device according to claim 18, wherein said first threshold is equal to an angle of between 110 and 150 degrees.

20. A device according to claim 18, wherein said detector means are made and arranged for controlling triggering the alarm when the orientation variation of the plate exceeds a second threshold, limited to approximately 90 degrees, by rotation around a transverse axis perpendicular to the infant's vertebral column.

21. A device according to claim 11, wherein said detector means are associated in said first unit of the device with means of attachment to the clothing of an infant to be supervised in said predetermined arrangement in the area of its navel.

22. A device according to claim 19, wherein said detector means are made and arranged for controlling triggering the alarm when the orientation variation of the plate exceeds a second threshold, limited to approximately 90 degrees, by rotation around a transverse axis perpendicular to the infant's vertebral column.
